(19) United States
(12) Patent Application Publication
(10) Pub. No.: US 2015/0366488 A1
(43) Pub. Date: Dec. 24, 2015

(54) Spinal motion sensor

(71) Applicant: Michael Anderson, Lindsay, CA (US)

(72) Inventor: Michael Anderson, Lindsay, CA (US)

(21) Appl. No.: 14/663,735

(22) Filed: Mar. 20, 2015

Related U.S. Application Data
(60) Provisional application No. 61/929,201, filed on Jan. 20, 2014.

(51) Int. Cl. A61B 5/11; A61B 5/00

(52) U.S. Cl. CPC A61B 5/1116; A61B 5/4566; A61B 5/4833; A61B 5/7445; A61B 5/6838; A61B 5/1121; A61B 5/486; A61B 5/7405; A61B 5/746; A61B 5/1123; A61B 2562/16

(57) ABSTRACT

A spinal motion sensor comprised of a system and method which detects any bending or twisting motion in the user's back. According to the method of the invention, the system alerts the user that the motion may result in a harmful injury. The device is worn on the belt of the user and features one or more inertial sensors, which detect the position of the user's back. Additional attitude sensors may be configured to be worn on the user's upper back or shirt collar, to determine the position of the back at additional locations. According to the method of the invention, the device presents an audible warning when the user moves into an inappropriate position.

### CROSS-REFERENCE TO RELATED APPLICATIONS

This application claims the benefit of U.S. Provisional Application 61/929,201, filed Jan. 20, 2014, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to medical monitoring devices and accessories, and in particular to a spinal motion sensor. Over 1.2 million back surgeries are being performed every year in the United States, often with lengthy post-operative physical therapy and recuperation. The most frequent post-operative instruction given by surgeons in these cases is to avoid any bending or twisting of the spine. Unfortunately, such motions are extremely common, everyday actions and there is no way for the patient to know when a bending or twisting motion has started. Prior to surgery, back pain provides ample notice to the user when a bending or twisting motion has started. But the surgery generally eliminates this pain, and old habits of bending and twisting usually return quickly, to the patient's detriment. A spinal motion sensor, which detects when the user has started a bending or twisting motion of the spine and alerts the user, would resolve this problem.

### SUMMARY OF THE INVENTION

Accordingly, the invention is directed to a spinal motion sensor and an associated system and method. According to the method of the invention, the device detects any bending or twisting motion of the user's back and alerts the user that the motion may result in a harmful position. The device is worn on the belt of the user and features one or more inertial sensors, which detect the position of the user's back. Additional attitude sensors may be configured to be worn on the user's upper back or shirt collar, to determine the position of the back at additional locations. According to the method of the invention, the device presents an audible warning when the user moves into an inappropriate position, defined as the user's back reaching an excessive degree of bend or an excessive degree of twist, as measured by the sensors. The device may be configured with standard concerns for good posture and undue back strain, or it may be configured by a user or the user's health care provider to test for particular motions which create a unique risk due to the user's particular injury or surgical recovery.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrates two embodiments of the invention and, together with the description, serve to explain the principles of the invention.

FIG. 1 is a side perspective view of the first exemplary embodiment, displaying the device 10, the belt clip 11, the face 12, and the control dial 13.

FIG. 2 is a side view of the first exemplary embodiment, displaying the device 10, the belt clip 11, the face 12, and the control dial 13.

FIG. 3 is a front view of the first exemplary embodiment, displaying the device 10, the face 12, the hands 12A, and the indicia 12B.

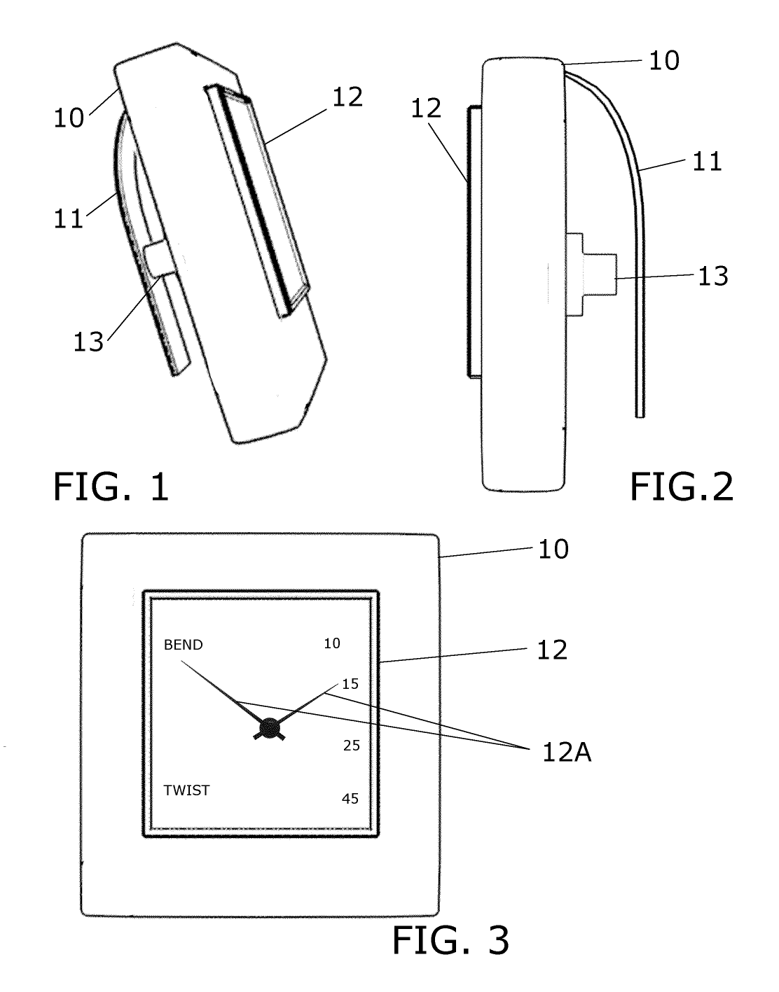

### DETAILED DESCRIPTION OF THE INVENTION

Referring now to the invention in more detail, the invention is directed to a spinal motion sensor and an associated system and method. According to the method of the invention, the device 10 detects any bending or twisting motion of the user's back and alerts the user that the motion may result in a harmful position. The device 10 is worn on the belt of the user and features one or more inertial sensors, which detect the position of the user's back. Additional attitude sensors may be configured to be worn on the user's upper back or shirt collar, to determine the position of the back at additional locations. The device 10 presents an audible warning when the user moves into an inappropriate position. The device 10 may be configured with standard concerns for good posture and undue back strain, or it may be configured by a user or the user's health care provider to test for particular motions which create a unique risk due to the user's particular injury or surgical recovery.

The first exemplary embodiment features a device 10 with a belt clip 11 extending downward from the upper edge of the rear surface. The front surface of the device 10 features a large face 12, resembling a clock face in that it displays two hands 12A. The hands 12A are rotatably affixed to the face 12 and electromechanically actuated in the manner of clock hands, and point to printed indicia 12B of the type of motion which the user has engaged in, such as bending or twisting, as well as the angular degree of the motion. On the rear surface, beneath the belt clip 11, is a control dial 13. The control dial 13 serves two functions. First, the control dial 13 features markings which correspond to angles of motion which may be indicated by the hands 12A on the face 12, and the control dial 13 may be set to one of these markings, to set the audible warning to activate when the marked angle of motion has been reached. Second, the control dial 13 provides a firm point of contact which may be used, in conjunction with the belt clip 11, to firmly secure the device 10 to the user's belt.

Internally, the device 10 features one or more inertial sensors, which detect the position of the user's back. The sensors are in electronic communication with the arms 12A such that the arms 12A are configured for outputting the degree of rotation of the back sensed by the sensors. Additional attitude sensors may be configured to be worn on the user's upper back or shirt collar, or elsewhere on the user's upper body, to determine the position of the back at additional locations. The attitude sensors may be temporarily affixed to the user's skin with an adhesive, or affixed to the user's clothing. Such additional attitude sensors are understood as external to the device 10 and in wireless electronic communication with the device 10. Preferably, the additional attitude sensors feature a radio frequency (RF) transmitter and a small battery, and transmit data to the device 10 identifying the motion of the user's back. The device 10 preferably features an RF receiver, a rechargeable battery, a microprocessor, a memory device, one or more inertial sensors, and a universal service bus (USB) port as its electronic components. The USB port enables the user or the user's health care provider to configure the device 10, to present an audible warning in response to the specific types and ranges of motion which are harmful to the user and would delay the user's recovery. A mobile application and a computer program are preferably provided, to enable the user or the user's health care provider to configure the device 10 using a smartphone, tablet, laptop, or desktop computer. In alternative embodiment, no additional attitude sensors or RF receiver are provided, and the device 10 is understood as confined within its single housing of the device 10, as shown, but remaining functional with no external components.

To use the first exemplary embodiment, the user or the user's health care provider downloads the mobile application into a smartphone or tablet, or the computer program into a laptop or desktop computer. The user then connects the smartphone, tablet, laptop or desktop computer to the device 10 via the USB port, using a cable which is provided, and configures the device 10 to present an audible warning in response to specific types and ranges of motion. Generically, this is understood as the step of programming the device 10 for specific types and ranges of motion, according to the method of the invention, and, generically, the device 10 is understood to be programmable in the sense that such limits may be entered. The user or the user's health care provider may then affix one or more attitude sensors to the user's skin, or the user's clothing, to supplement the internal inertial sensor or sensors of the device 10. The user may use the control dial 13 to adjust the configuration of the device 10. The user then affixes the device 10 to the user's belt, using the belt clip 11, at a predetermined position relative to the user's spine. The user may then move naturally while monitoring the device 10 for excessive bending or twisting of the spine, and limiting his or her movements accordingly.

The device 10 is preferably manufactured from rigid, durable materials such as plastic, acrylic polymers, steel, aluminum alloy, and copper alloys. Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A spinal motion sensor system and method, the system comprising:
(a) a device;
(b) said device having a face;
(c) said device having a plurality of indicia printed on said face;
(d) a pair of hands;
(e) said pair of hands being rotatably affixed to said face in the manner of clock hands;
(f) said pair of hands being electromechanically actuated;
(f) said device containing at least one sensor;
(g) said at least one sensor being in electronic communication with said pair of hands;
(h) a belt clip;
(i) said belt clip being affixed to said device;
(j) a human user;
(k) said device being affixed to the waist of said human user via said belt clip;
(l) said device being configured such that said at least one sensor outputs the angle of bending of the spine of said human user;
(m) said device being configured such that one of said hands indicates, by pointing to said indicia, the angle of bending of the user's spine;
(n) said device being configured such that said at least one sensor outputs the angle of twisting of the spine of said human user;
(o) said device being configured such that one of said hands indicates, by pointing to said indicia, the angle of twisting of the user's spine;
and the method comprising:
(j) said human user affixing said device to the belt of said human user via said belt clip;
(k) said human user proceeding to move naturally;
(l) said human user monitoring said device for excessive bending or excessive twisting; and
(m) limiting the movements of said human user accordingly.

2. The spinal motion sensor system and method of claim 1, the method further comprising presenting, via said device, an audible alarm in response to said human user's backing reaching an excessive degree of bend or an excessive degree of twist, as measured by said at least one sensor.

3. The spinal motion sensor system and method of claim 1 wherein said at least one sensor comprises at least one additional sensor; said at least one additional sensor being external to said device and in electronic communication with said device.

4. The spinal motion sensor system and method of claim 2 wherein said at least one sensor comprises at least one additional sensor; said at least one additional sensor being external to said device and in electronic communication with said device.

5. The spinal motion sensor system and method of claim 1, the method further comprising programming said device for specific types and ranges of motion; said device being configured so as to be programmable therefor.

6. The spinal motion sensor system and method of claim 2, the method further comprising programming said device for specific types and ranges of motion; said device being configured so as to be programmable therefor.

7. The spinal motion sensor system and method of claim 3, the method further comprising programming said device for specific types and ranges of motion; said device being configured so as to be programmable therefor.

8. The spinal motion sensor system and method of claim 4, the method further comprising programming said device for specific types and ranges of motion; said device being configured so as to be programmable therefor.

9. The spinal motion sensor system and method of claim 1 wherein said device is confined within its single housing.

10. The spinal motion sensor system and method of claim 2 wherein said device is confined within its single housing.

11. The spinal motion sensor system and method of claim 5 wherein said device is confined within its single housing.

12. The spinal motion sensor system and method of claim 6 wherein said device is confined within its single housing.

13. The spinal motion sensor system and method of claim 7 wherein said device is confined within its single housing.

14. The spinal motion sensor system and method of claim 8 wherein said device is confined within its single housing.
